(19) United States
(12) Patent Application Publication
(10) Pub. No.: US 2018/0187273 A1
(43) Pub. Date: Jul. 5, 2018

(54) Universal Priming Mixture for Amplification of Influenza Viral Gene Segments for Diagnostic Applications

(71) Applicant: InDevR Inc., Boulder, CO (US)

(72) Inventors: Amber W. TAYLOR, Boulder, CO (US); Erica Dawson TENENT, Boulder, CO (US); Kathy L. ROWLEN, Boulder, CO (US)

(21) Appl. No.: 15/740,761

(22) PCT Filed: Jun. 30, 2016
(86) PCT No.: PCT/US16/40565

§ 371 Date: Dec. 28, 2017

Related U.S. Application Data
(60) Provisional application No. 62/188,099, filed on Jul. 2, 2015.

(51) Int. Cl. C12Q 1/70; G01N 33/569; C12Q 1/686

(52) U.S. Cl. CPC C12Q 1/701; G01N 33/56983; G01N 2333/11; C12Q 2600/16; C12Q 2600/112; C12Q 1/686

(57) ABSTRACT

Provided herein is a multiplexed RT-PCR based assay for amplification multiple whole gene segments of influenza A and B. Accordingly, various oligonucleotide primers are disclosed, including for use in a multiplex RT-PCR process for influenza detection and characterization. The primers and instructions for use may be provided in the form of a kit. Applications for the primers and related methods include as a clinical virology tool, epidemiological and surveillance tool, as well as in animal surveillance testing for influenza viruses.

### CROSS-REFERENCE TO RELATED APPLICATIONS

This application claims the benefit of priority to U.S. Provisional Patent Application No. 62/188,099 filed on Jul. 2, 2015, which is specifically incorporated by reference to the extent not inconsistent herewith.

### STATEMENT REGARDING FEDERALLY SPONSORED RESEARCH OR DEVELOPMENT

This invention was made with Government support under Grant/Contract No. A1077112 awarded by NIH/NIAID. The Government may have certain rights in the technology of this patent application.

### REFERENCE TO A SEQUENCE LISTING

A sequence listing containing SEQ ID NOs: 1-13 is provided herewith and is specifically incorporated by reference.

### BACKGROUND OF INVENTION

Provided herein is a unique mixture of primer sets that provide universal amplification of select key gene segments within the influenza virus genome. This carefully balanced mixture of primer sets allow for reliable and robust detection and characterization of influenza virus within a single reaction.

Although other purported universal primer sets have been used to amplify certain influenza virus gene segments, none have demonstrated sufficient robustness for use in detailed characterization of all influenza viruses without resorting to full genome sequencing. Accordingly, there is a need in the art for a universal primer set to amplify influenza virus gene segments in a manner that is sufficiently robust to provide detailed characterization of a wide range of influenza viruses without full genome sequencing.

### SUMMARY OF THE INVENTION

Provided herein is a carefully balanced and designed mixture of primer sets to ensure universal amplification of key gene segments within the influenza virus genome. Specifically, the primer set robustly amplifies whole hemagglutinin (HA), neuraminidase (NA), matrix (M), non-structural (NS), and nucleoprotein (NP) gene segments for influenza A, whole HA and NA gene segments for influenza B, and includes an internal positive control primer set based on the gene that codes for the 18s rRNA compatible with all eukaryotic samples. Furthermore, the primer set is compatible with a multiplex test, where amplification of the different gene segments occurs in parallel within a single test reaction. Such a primer set has use in range of applications, including diagnostics and characterization of potentially pandemic influenza virus, including for influenza A and, as discussed herein, influenza B.

There is large amount of information around the influenza genome that is harnessed herein to specially design primers to amplify each targeted gene segment in its entirety. In particular, provided herein are gene segment-specific primers around the highly conserved 13 nt and 12 nt regions of the 5′ and 3′ ends of each gene segment within the influenza A genome. A similar region also exists for influenza B. By targeting these conserved regions, the potential for RT-PCR failure due to genetic mutation over time is minimized, thus maintaining the validity of the approach described herein over time, including during potential future threats. The RT-PCR reaction conditions are optimized with respect to Mg²⁺, RT time and temperature, RT inactivation/Taq activation time, melting time, annealing temperature and time, and extension temperature and time, specifically for multiplex amplification of Flu A HA, NA, M, NS, NP; Flu B HA, NA; and an 18s RNA internal control.

An important aspect of the methods and underlying primers is the ability to rapidly and reliably characterize influenza in a single shot approach, referred herein as a multiplex test. In this manner, simultaneous amplification of nucleotide targets by the different primer pairs occurs within a single tube.

Provided herein is an isolated and purified nucleic acid for use in influenza detection, including one or more nucleic acid sequences of the following:

SEQ ID NO:1 (AGAGCRAAAGCAGGTAG) and/or SEQ ID NO:2 (GGGAGTAGAAACAAGGTAG) for targeting an influenza A whole M gene segment, wherein the nucleotide R is a purine;

SEQ ID NO:3 (AGAGCAAAAGCAGGAG) and/or SEQ ID NO:4 (GGGAGTAGAAACAAGGAG) for targeting an influenza A whole NA gene segment (subtype N1, N2, N4, N5, N8);

SEQ ID NO:5 (AGAGCAAAAGCAGGTG) and/or SEQ ID NO:6 (GGGAGTAGAAACAAGGTG) for targeting an influenza A whole NA gene segment (subtype N3);

SEQ ID NO: 7 (GAGCRAAAGCAGGGT) and/or SEQ ID NO:8 (GGGAGTAGAAACAAGGGT) for targeting an influenza A whole NA, NS, NP gene segments (subtype N6, N7, N9) wherein the nucleotide R is a purine; and

SEQ ID NO: 9: (AGCAAAAGCAGGGG) and/or SEQ ID NO:8 for targeting an influenza A whole HA gene segment.

In an aspect, provided are any one or more of the pairs of forward/reverse primers described herein that are useful in amplification of corresponding targets of an influenza gene, including one or more pairs of SEQ ID NOs: 1 and 2; SEQ ID NOs: 3 and 4; SEQ ID NOs: 5 and 6; SEQ ID NOs: 7 and 8; SEQ ID NOs: 9 and 8; and any combinations thereof.

An advantage of the primers provided herein is that they are configured for use in a multiplex manner, for substantially simultaneous amplification of multiple amplicons in a single sample.

In addition, any of the primers and paired forward/reverse primers of SEQ ID NOs: 1-9 may be further used in a multiplex manner with primers used to detect other influenza virus types, strains, or mutations. For example, the primers may be used in a multiplex assay for detection of influenza A and/or influenza B virus. In this aspect, the isolated and purified nucleic acid may further comprise at least one additional nucleic acid for targeting an influenza B gene, such as SEQ ID NO: 10 (AGCAGAAGCAGAGCAT) and/or SEQ ID NO: 11 (CAGTAGTAACAAGAGCATTT) for targeting an influenza B whole HA and NA gene.

Any of the isolated and purified nucleic acid described herein may further comprise at least one additional nucleic acid that is a control, useful as an internal control for experimental validation, such as appropriate sample handling, processing and amplification. In this manner, should there be no detectable amplicons the control will confirm that amplification has indeed occurred for the sample and increase confidence that lack of relevant amplicons is due to lack of virus sample and not an error that hinders or prevents proper amplification. An example of primers useful as an internal control is SEQ ID NO: 12 (CCTGAGAAACGGCTAC) and/or SEQ ID NO: 13 (TTATGGTCGGAACTACG) for targeting a gene coding for 18s rRNA. The control primers are designed for compatibility with a multiplex test so that all amplicons are generated from all the relevant primers in a single shot from a single test sample.

Any of the isolated and purified nucleic acids described herein may further comprise one or more non-natural modifications, such as phosphorylation at a 5′-end of the nucleic acid.

As described, the isolated and purified nucleic acid of the invention may comprise any subset and combination of the primers described herein. For example, any of the primers described herein may be explicitly excluded from a combination of primers, such as a combination of primers without SEQ ID NO:9.

Similarly, the isolated and purified nucleic acid may comprise each of SEQ ID NOs: 1-9 for targeting HA, NA and M of influenza A.

In an embodiment, the isolated and purified nucleic acid comprises SEQ ID NO: 2.

The invention provided herein may comprise a plurality of primers, such as comprising each of SEQ ID NOs:1-13, or any subcombinations thereof. Any of the primers or plurality of primers may be configured for use in a single multiplex RT-PCR, such as by a premixed cocktail of primers contained in a single mixture.

Also provided herein is a universal primer set for amplification of whole gene segments HA, NA, M, NS and NP from an influenza A virus in a single multiplex reaction. In this aspect, the universal primer set may comprise isolated and purified nucleic acids of SEQ ID NOs:1-9 for targeting the respective whole gene segments.

In an embodiment, a universal primer set may comprise a plurality of nucleic acid primers for amplification of whole gene segments HA and NA from an influenza B virus in a single multiplex reaction, such as a plurality of nucleic acid primers comprising isolated and purified nucleic acids of SEQ ID NOs:10-11.

In an embodiment, a universal primer set may comprise a plurality of nucleic acid control primers for amplification of a control 18s gene in a single multiplex reaction, such as a plurality of nucleic acid control primers comprising isolated and purified nucleic acids of SEQ ID NOs:12-13.

In an embodiment, a universal primer set is provided for amplification of whole gene segments HA and NA from an influenza B virus in a single multiplex reaction, the universal primer set comprising isolated and purified nucleic acids of SEQ ID NOs:10-11 for targeting the whole gene segments. The universal primer set may further comprise SEQ ID NOs:12-13 for amplification of a control 18s gene.

In an embodiment, the universal primer set may combine each of the above-reference universal primer sets, so that primers corresponding to SEQ ID NOs: 1-13 are provided for a single multiplex reaction related to target gene segments from influenza A, influenza B, and an internal control.

Any of the primers and universal primer sets may be described in terms of one or more functional parameters, including those that impact the ability to achieve a reliable and robust multiplex reaction for influenza characterization.

For example, individual nucleic acids may be described in terms of a selected concentration for substantially simultaneous amplification of all influenza viruses within a single sample (e.g., a multiplex reaction), wherein the concentration of each primer is within 25% of a concentration value selected from one or more of: SEQ ID NO:1 having the concentration value of 300 nM (225 nM to 375 nM); SEQ ID NO:2 having the concentration value of 300 nM (225 nM to 375 nM); SEQ ID NO:3 having the concentration value of 400 nM (300 nM to 500 nM); SEQ ID NO:4 having the concentration value of 400 nM (300 nM to 500 nM); SEQ ID NO:5 having the concentration value of 400 nM (300 nM to 500 nM); SEQ ID NO:6 having the concentration value of 400 nM (300 nM to 500 nM); SEQ ID NO:7 having the concentration value of 400 nM; SEQ ID NO:8 having the concentration value of 500 nM (375 nM to 625 nM); SEQ ID NO:9 having the concentration value of 500 nM (375 nM to 625 nM); SEQ ID NO:10 having the concentration value of 400 nM (300 nM to 500 nM); SEQ ID NO:11 having the concentration value of 400 nM (300 nM to 500 nM); SEQ ID NO:12 having the concentration value of 80 nM (60 nM to 100 nM); SEQ ID NO:13 having the concentration value of 80 nM (60 nM to 100 nM); or any combination thereof. The primers may be provided separately with mixing instructions to achieve a desired primer concentration for each primer, or may be provided as a premixed cocktail with appropriate relative amounts of primers ready for use or at a higher “stock” concentration ready for dilution ahead of use.

Another useful functional description is a melting temperature, such as the temperature at which a primer is calculated to release from its target sequence. For example, each individual nucleic acid has a melting temperature that is substantially matched to every other individual nucleic acid melting temperature for a multiplex reaction, to ensure the reaction containing the primer population may occur in a single sample without the need for separate thermal cycling conditions. Accordingly, in this context “substantially matched” refers to a melting temperature of a primer that is selected so as to achieve detectable amplification during a multiplex PCR along with other primers present with a resultant plurality of amplicons from distinct primer pairs by a single thermal cycling protocol. Melting temperatures of short nucleic acid sequences can be calculated in a variety of ways, including by the use of a nearest neighbor calculation to estimate the thermodynamic parameters that are then used to predict the melting temperature based on a 2-state model. In this aspect, the melting temperature may be characterized by one or more of: a minimum primer melting temperature that is between about 51.0-52.0° C. and a maximum primer melting temperature is between about 53.7-53.9° C.; an average melting temperature that is between about 52.5-52.7° C.; or a standard deviation of all the melting temperatures that is less than about 1° C. Exemplary melting temperatures for each of the SEQ ID NOs: 1-13 include, respectively (in ° C.), 52.4, 51.1, 51.7, 51.8, 52.6, 52.6, 53.8, 53.7, 53.3, 52.1, 49.5, 51.9, 52.4, with an attendant average ±SD or 52.6±0.9.

Also provided herein are various methods of using any one or more of the primers described herein. For example, the method may be for determining the presence or absence of influenza virus in a sample by providing a universal primer set or cocktail for amplification of influenza whole gene targets. The universal primers may comprise: one or more influenza A gene segments HA, NA, M, NS and NP; and/or one or more influenza B gene segments HA and NA. A sample is contacted with the universal primer cocktail and RT-PCR performed on the sample in contact with the universal primer cocktail in a single multiplex reaction step. Amplified products or amplicons are detected from the performing RT-PCR step, thereby determining the presence or absence of influenza virus.

Any of the methods may further use a universal primer cocktail comprising primers for amplification of an 18s control. The 18s control may amplify a variety of eukaryotic species to facilitate testing of influenza virus in a range of species, including human, pig, bird, horse, dog, cat, and other influenza animal reservoirs.

In an aspect the universal primer cocktail comprises a plurality of primers corresponding to SEQ ID NOs: 1-13, thereby providing potential for influenza A, influenza B and internal control amplicons.

The performing step may be described in terms of temperatures, times and cycling conditions, and the methods provided herein are compatible with a range of temperatures, times, and cycling conditions, depending, for example, on PCR kit components and reagents, geometry, concentration and other experimental conditions that are known to affect PCR. One example of such a cycling condition includes: a reverse transcription (RT) step at a temperature of between 47° C. to 49° C. for a time of between 18 minutes and 22 minutes; a RT inactivation and polymerase activation step at a temperature of between 93° C. and 95° C. for between 2 min and 4 min; a polymerase chain reaction (PCR) step for a PCR cycle number, wherein the PCR cycle number is greater than or equal to 35 cycles and less than or equal to 45 cycles.

The methods provided herein, depending on the application of interest, may be used to detect influenza B, seasonal A/H1N1, seasonal A/H3N2, and non-seasonal A influenza strains.

Also provided are kits for detecting an influenza virus, such as a kit comprising: any combination of the primers described herein, wherein the primers comprise a plurality of forward primers and a plurality of reverse primers for amplification of a plurality of influenza whole gene segments selected from the group consisting of influenza HA, NA, M, NS and NP; and reagents for amplification by RT-PCR of the plurality of whole gene segments in a single multiplex reaction.

In another aspect, provided is a kit for carrying out any of the methods described herein, such as a kit comprising: at least one container containing a plurality of distinct oligonucleotide primers having individual sequences that consist of SEQ ID NOs: 1-13; at least one container containing reagents for performing RT-PCR for amplification of whole gene segments for influenza HA, NA, M, NS, and NP; and instructions for performing RT-PCR using the oligonucleotide primers and reagents in a single multiplex reaction and detecting amplification products.

Without wishing to be bound by any particular theory, there may be discussion herein of beliefs or understandings of underlying principles relating to the devices and methods disclosed herein. It is recognized that regardless of the ultimate correctness of any mechanistic explanation or hypothesis, an embodiment of the invention can nonetheless be operative and useful.

### BRIEF DESCRIPTION OF THE DRAWINGS

FIG. 1. Influenza primers of the instant disclosure with consensus sequences of the segments of interest used in the design of the primers. The top panel are the forward primers (SEQ ID Nos: 1, 3, 5, 7, 9 and 10). The bottom panel contains the reverse primers (SEQ ID Nos: 2, 4, 6, 8 and 11).

FIG. 2. Internal control forward (SEQ ID NO: 12) and reverse (SEQ ID NO: 13) primer, with a 95% consensus sequence from human, horse, chicken and wild swine aligned thereto.

FIG. 3. Gel electrophoresis results confirming amplification of NS, NP and NA gene segments for N6, N7 and N9 viruses by primers corresponding to SEQ ID NO: 7 and 8.

FIG. 4. Line scan to quantify relative amount of amplification products from the gel of FIG. 3.

FIG. 5. Agarose gel electrophoresis on amplification products from an A/H1N1 virus (H1), an A/H3N2 virus (H3), an influenza B virus (B), and a negative control (Neg) in a singleplex manner, with the relevant primers indicated by SEQ ID NOs.

FIG. 6. Effect of changing concentration of the internal control primers on amplification of influenza in a multiplex test. Three viruses were tested (A/H1N1, A/H3N2, B virus and negative control) at 150 nM, 100 nM, 0 nM and 50 nM internal primer control concentration.

FIG. 7. Effect of annealing temperature on amplicon detection for various template RNA concentrations ranging from 10³ to 10⁶ copies/mL.

FIG. 8. Gel electrophoresis detection of flu A and B gene segments by a single multiplex RT-PCR reaction, using a stock primer mix.

FIG. 9. Gel electrophoresis detection of various virus samples from a multiplex RT-PCR reaction, with the amplicons corresponding to gene segments for HA, NP, NA, M, NS and 18s as indicated.

FIG. 10 summarizes various physical parameters associated with the various primers of a universal primer set.

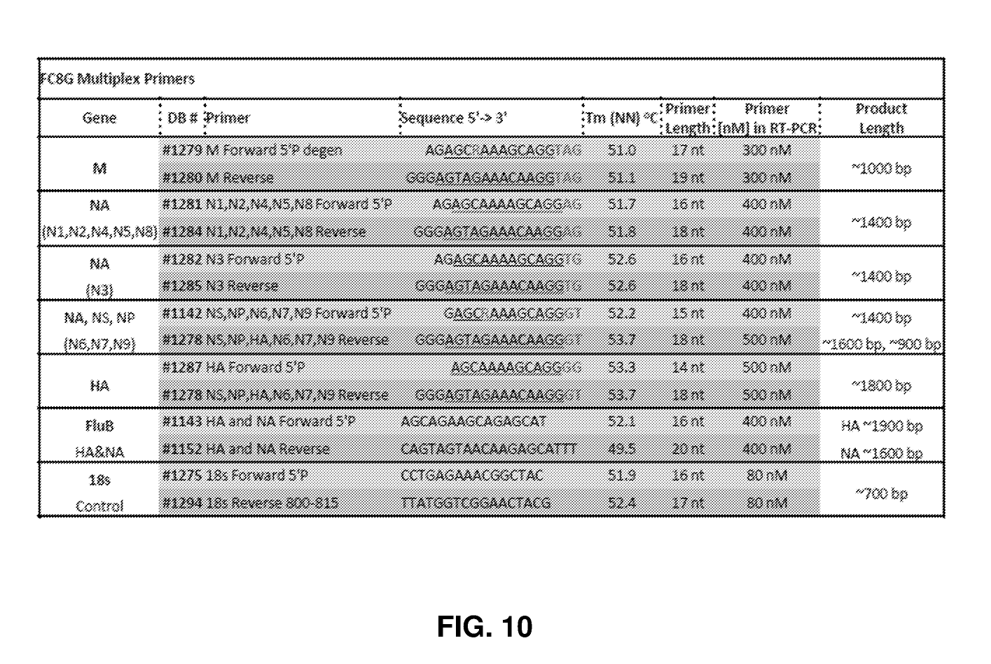

FIG. 11 Microarray images for the detection of different influenza A subtypes after multiplex RT-PCR amplification with SEQ ID Nos: 1-13 and subsequent hybridization to the chip. Upper left image represents a negative sample in which influenza virus was not present in which only controls and fiducial markers show resulting fluorescence signal on the microarray. The remaining images demonstrate applicability of the universal primers for various influenza subtypes, with subtypes H5N2, H11N3, H4N6, H7N7, H1N1 specifically exemplified.

### DETAILED DESCRIPTION OF THE INVENTION

In general, the terms and phrases used herein have their art-recognized meaning, which can be found by reference to standard texts, journal references and contexts known to those skilled in the art. The following definitions are provided to clarify their specific use in the context of the invention.

“Target” refers to the nucleic acid sequence of the influenza virus of interest.

“Sample” refers to a biological material that may be tested for the presence of an influenza virus. The sample may be biological material from a human or a non-human animal such as a specimen, material grown in cell culture, egg culture, or grown by other methods, or environmental materials that may be suspected of containing influenza. Exemplary samples include, but are not limited to, nasopharyngeal swabs, nasopharyngeal aspirates, nasal swabs or washes, throat swabs, oropharyngeal swabs or washes, tracheal aspirates, broncheoalveolar lavage, sputum, saliva, tissue samples, cell cultured material, egg cultured material, blood, plasma, serum, mucus, cloacal swabs, and the like.

“Primer” refers to an oligonucleotide complementary to and capable of selective binding to the cDNA or RNA target molecule and provides the 3′-OH-end of a substrate to which a polymerase can add nucleotides of a growing DNA chain in the 5′ to 3′ direction.

“RT-PCR” refers to reverse transcription polymerase chain reaction and is used to detect specific RNA, in this case specific gene segments of the influenza virus genome, such as by reverse transcribing the RNA of interest into its DNA complement through the use of reverse transcriptase. The newly synthesized cDNA can be amplified using traditional PCR. In an aspect, the RT-PCR provided herein is by a one-step approach, wherein the entire reaction from cDNA synthesis to PCR amplification occurs in a single tube. Alternatively, the process described herein is compatible with a two-step reaction requires that the reverse transcriptase reaction and PCR amplification be performed in separate tubes.

“PCR solution” refers to materials required to perform a PCR as known in the art. Examples of such materials include primers, enzymes such as polymerases (e.g., Taq polymerase), dNTP, nuclease inhibitors, salts (MgCl₂), PCR buffers, and other additives including but not limited to betaine, DMSO, formamide, detergents, and tetramethylammonium chloride to facilitate effective PCR. The PCR solution may contain nucleic acid material from a biological cell, viral particle, or other appropriate biological material, such as nucleic acid material from lysed cells or virus particles. PCR product refers to the nucleic acid that is produced as a result of the polymerase chain reaction process.

“Singleplex” refers to an RT-PCR or PCR reaction that is carried out using a single pair of forward and reverse primers. For the purposes of the current invention, singleplex may refer to a reaction in which a single primer pair amplifies one or more gene segments from the same starting material, but does not refer to a reaction in which more than 1 primer pair is used simultaneously in the same RT-PCR or PCR reaction tube.

“Multiplex”, in contrast, refers to the use of more than one pair of primers intended to amplify multiple target gene segments simultaneously within a single tube. In this manner, all the primers may be contained within one tube to which a sample is introduced or positioned. All desired Flu A, Flu B and internal control gene segments are then amplified via the plurality of forward and reverse primers within the tube.

“Universal primer set” or “universal primer cocktail” refers to nucleotide sequences designed to detect any influenza virus by detection of specific gene segments within the influenza genome by RT-PCR.

As used herein, the terms “isolated and/or purified” refer to in vitro isolation of a RNA or DNA molecule from its natural cellular environment, and from association with other components of the cell, such as adjacent nucleic acid, so that it can be sequenced, replicated, expressed and/or used, such as a primer in a PCR method.

An “isolated and purified nucleic acid molecule” is a nucleic acid the structure of which is not identical to that of any naturally occurring nucleic acid. This term covers, for example, DNA which has part of the sequence of a naturally occurring genomic DNA, but does not have the flanking portions of DNA found in the naturally occurring genome. The term also includes, for example, a nucleic acid having non-natural or non-native modification, including 5′ modifications, such as phosphorylation.

Some nucleic acid sequence variation of SEQ ID NOs: 1-13 is tolerated without loss of function. In fact, some nucleic acid variation is expected and understood in the art, without substantially affecting primer function.

A variant nucleic acid sequence of the invention has at least about 80%, more preferably at least about 90%, and even more preferably at least about 95%, but less than 100%, contiguous nucleic acid sequence identity to a nucleic acid sequence comprising any of SEQ ID NOs: 1-13, or a fragment thereof. However, these nucleic acid sequences still provide a functional primer specific for the target gene, as assessed by a detectable amplicon under a multiplex reaction test condition. The nucleic acid similarity (or homology) of two sequences can be determined manually or using computer algorithms well known to the art.

The term “sequence homology” or “sequence identity” means the proportion of base matches between two nucleic acid sequences. When sequence homology is expressed as a percentage, e.g., 50%, the percentage denotes the fraction of matches over the length of sequence that is compared to some other sequence. Gaps (in either of the two sequences) are permitted to maximize matching; gap lengths of 15 bases or less are usually used, 6 bases or less are preferred with 2 bases or less more preferred. When using oligonucleotides as probes or primers, the sequence homology between the target nucleic acid and the oligonucleotide sequence is generally not less than 17 target base matches out of 20 possible oligonucleotide base pair matches (85%); preferably not less than 9 matches out of 10 possible base pair matches (90%), and more preferably not less than 19 matches out of 20 possible base pair matches (95%).

The invention further includes isolated and purified DNA sequences which hybridize under standard or stringent conditions to the target nucleic acid sequences by the sequences of the present invention, including complements of isolated and purified DNA sequences that hybridize under standard or stringent conditions to any of the primer sequences provided herein. Hybridization procedures are useful for identifying polynucleotides with sufficient homology to the subject sequences to be useful as taught herein. The particular hybridization techniques are not essential to the subject invention. As improvements are made in hybridization techniques, they can be readily applied by one of ordinary skill in the art. Preferably, the isolated nucleic acid molecule comprising the sequence given in any of SEQ ID NOs:1-13, a variant or a fragment thereof, e.g., a nucleic acid molecule that hybridizes under moderate, or more preferably stringent, hybridization conditions to the respective target sequence or a fragment thereof. Various degrees of stringency of hybridization can be employed. The more stringent the conditions, the greater the complementarity that is required for duplex formation. Stringency can be controlled by temperature, primer concentration, primer length, ionic strength, time, and the like. Preferably, hybridization is conducted under moderate to high stringency conditions by techniques well known in the art, as described, for example in Keller, G. H., M. M. Manak (1987) DNA Probes, Stockton Press, New York, N.Y., pp. 169-170, hereby incorporated by reference. For example, stringent conditions are those that (1) employ low ionic strength and high temperature for washing, for example, 0.015 M NaCl/0.0015 M sodium citrate (SSC); 0.1% sodium lauryl sulfate at 50° C., or (2) employ a denaturing agent such as formamide during hybridization, e.g., 50% formamide with 0.1% bovine serum albumin/0.1% Ficoll/0.1% polyvinylpyrrolidone/50 mM sodium phosphate buffer at pH 6.5 with 750 mM NaCl, 75 mM sodium citrate at 42° C. Another example is use of 50% formamide, 5×SSC (0.75 M NaCl, 0.075 M sodium citrate), 50 mM sodium phosphate (pH 6.8), 0.1% sodium pyrophosphate, 5 times Denhardt's solution, sonicated salmon sperm DNA (50 μg/ml), 0.1% sodium dodecylsulfate (SDS), and 10% dextran sulfate at 4° C., with washes at 42° C. in 0.2×SSC and 0.1% SDS.

An example of high stringency conditions is hybridizing at 68° C. in 5×SSC/5×Denhardt's solution/0.1% SDS, and washing in 0.2×SSC/0.1% SDS at room temperature. An example of conditions of moderate stringency is hybridizing at 68° C. in 5×SSC/5×Denhardt's solution/0.1% SDS and washing at 42° C. in 3×SSC. The parameters of temperature and salt concentration can be varied to achieve the desired level of sequence identity between primer and target nucleic acid. See, e.g., Sambrook et al. (1989) supra or Ausubel et al. (1995) Current Protocols in Molecular Biology, John Wiley & Sons, NY, NY, for further guidance on hybridization conditions.

In general, salt and/or temperature can be altered to change stringency. With a labeled DNA fragment >70 or so bases in length, the following conditions can be used: Low, 1 or 2×SSPE, room temperature; Low, 1 or 2×SSPE, 42° C.; Moderate, 0.2× or 1×SSPE, 65° C.; and High, 0.1×SSPE, 65° C.

“Complement” or “complementary sequence” means a sequence of nucleotides which forms a hydrogen-bonded duplex with another sequence of nucleotides according to Watson-Crick base-pairing rules. For example, the complementary base sequence for 5′-AAGGCT-3′ is 3′-TTCCGA-5′. This invention encompasses complementary sequences to any of the nucleotide sequences claimed in this invention.

### Example 1: Influenza Primer Design

Design of influenza primers involves downloading appropriate influenza sequences specific for each gene segment of interest for all available types and subtypes from Gen Bank. Sequences for each gene segment of interest are first aligned using Muscle in BioEdit® (version 7.0.9.0) to achieve a first approximation aligned sequence set, and alignments are also visually examined to ensure alignment at the conserved end regions of the gene segments. Accurate alignment at the sequence terminus can be challenging due to inconsistent and sometimes incomplete sequence information, and is important because without proper alignment at the sequence termini, an accurate consensus sequence cannot be generated. Once proper alignment of the sequence termini is achieved, consensus sequences (95%) are generated from each relevant sequence alignment. Care is taken to identify point mutations that could cause failure of the existing primer sets. Accordingly, appropriate primer variations are designed to overcome limitations and enhance amplification robustness. As discussed below, systematic studies are conducted to determine which primer sets, each with a specific concentration, provide reliable and robust amplification of all influenza viruses.

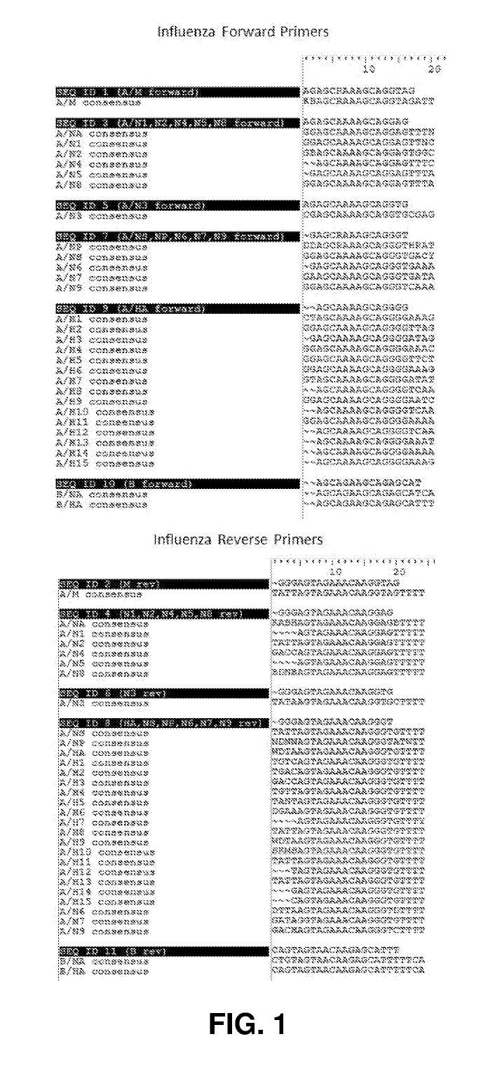

For HA and NA, consensus sequences are created for the gene segment overall (independent of subtype), as well as for each HA and NA subtype represented in the alignment, anticipating that full conservation for all subtypes may be impossible due to the high genetic diversity between subtypes. Sequence alignments are then examined in an attempt to design the minimum number of primer sequences required to amplify all gene segments of interest for all HA and NA subtypes. Primers are also designed to have melting temperatures as closely matched as possible to facilitate amplification in a single tube. Given the low prevalence of certain viral subtypes in certain alignments (for example, the comparatively low number of NA sequences from H5N1 viruses in the N1 alignment due to the high number of H1N1 viral sequences in the alignment), the primer sequences designed are checked against the sequences for low prevalence subtypes to ensure a good match and high probability for amplification of these low prevalence subtypes. In addition, potential primers are examined for primer cross-hybridization as well as potential for self-binding (loop formation). The influenza primers are summarized in (see, e.g., SEQ ID NOs: 1-11) FIG. 1 and TABLE 3 along with the consensus sequences for the influenza gene segments of interest utilized in their design.

### Example 2: Internal Control Primer Design

18s rDNA that codes for the rRNA is designed as the internal control. Given that 18s rDNA is present in all eukaryotes and would therefore be present in human respiratory specimens as well as appropriate specimens from other animals that experience influenza infections, we design primers to amplify a relatively well-conserved portion of the 18s rDNA in a number of relevant species. This approach facilitates use of the same pair of internal control primers to act as a process control check in assays anticipated for human use as well as those anticipated for use in animals including chickens, pigs, and horses (all of which also experience influenza infections).

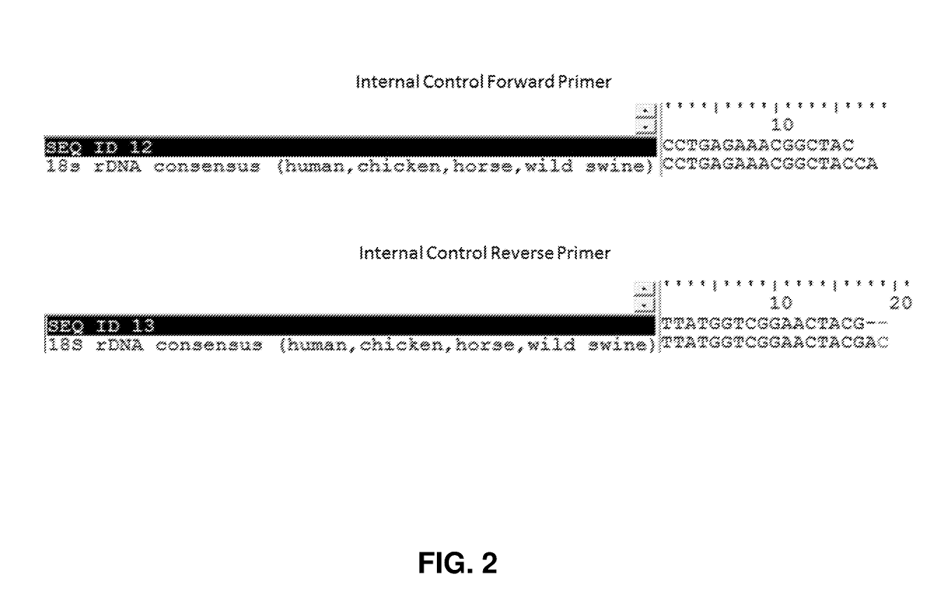

Sequence design involves downloading available 18s rDNA sequences from GenBank from relevant species. A 95% consensus sequence is determined from a database consisting of rDNA sequences from the following: 6 human sequences, 2 horse sequences, 4 chicken sequences, and 2 wild swine sequences. FIG. 2 shows the consensus sequence generated aligned against the forward and reverse primers designed (SEQ ID NO: 12 and SEQ ID NO: 13). Band lengths anticipated for all amplification products expected from the combination of SEQ IDs 1-13 are shown in TABLE 1.

### Example 3: Confirmation of Amplification of N6, N7, and N9 NA Due to Co-Amplification of NS Gene

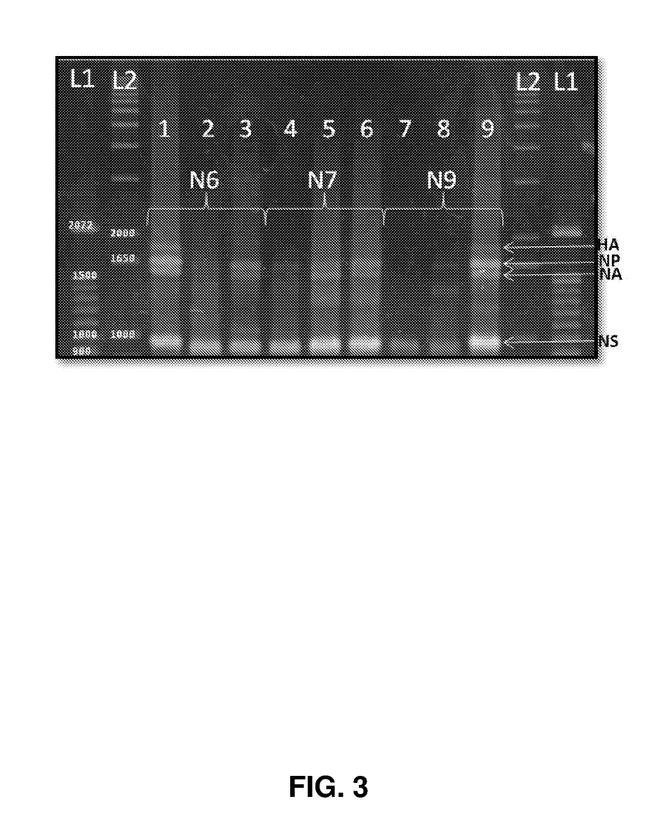

Based on sequence analysis, SEQ ID 7 and SEQ ID 8 are anticipated to co-amplify the NS and NP gene segments in addition to amplifying the NA gene segment for N6, N7, and N9 viruses. Given that the NS gene segment is significantly shorter (1000 bp), the ability of this single primer pair to effectively amplify the NA gene segment for N6, N7, and N9 viruses is tested. Gel electrophoresis results for this testing are shown in FIG. 3. Testing for N6 amplification is performed on extracted RNA from two H4N6 viruses (lanes 1 and 3 in FIGS. 3 and 1 H3N6 virus (lane 2). Testing for N7 amplification is performed on extracted RNA from two H10N7 viruses (lanes 4 and 5) and one H7N7 virus (lane 6). Testing for N9 amplification is performed on extracted RNA from an H10N9 virus (lane 7), an H11N9 virus (lane 8), and H2N9 virus (lane 9). Two different molecular weight ladders (labeled L1 and L2) are also shown, along with the expected lengths for the gene segments amplified.

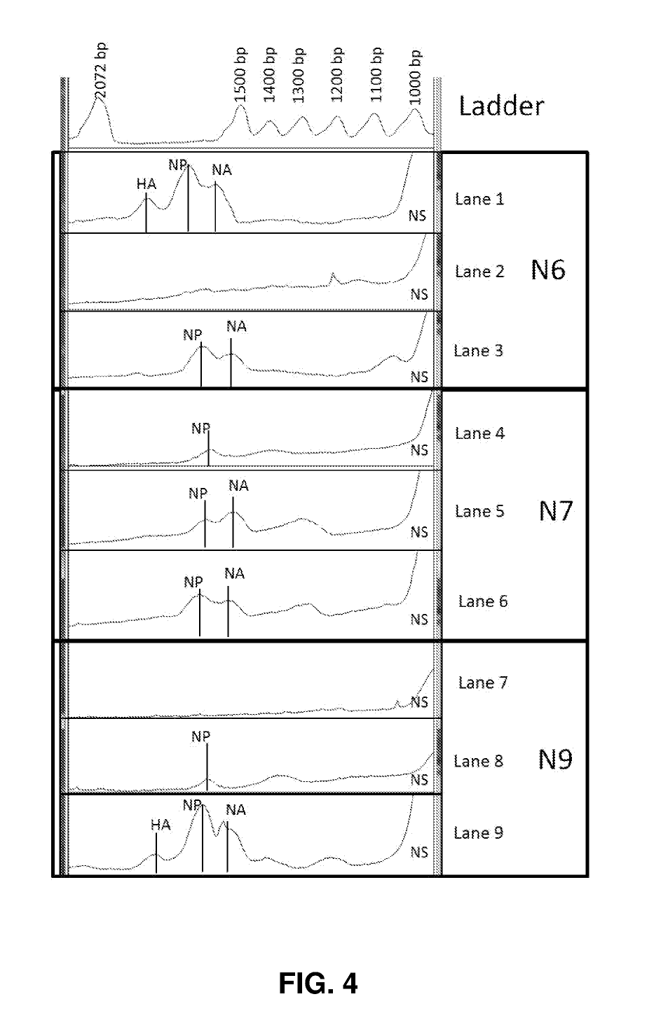

To examine more closely the relative amounts of the amplification products in the gel in FIG. 3, a line scan of each lane of the gel is taken to highlight the band intensities. The results of these line scans are shown in FIG. 4. Lanes 1, 3, 5, 6, and 9 show successful amplification of the NA gene segment for H6, H7, and H9 viruses. NA does not amplify in all cases, likely due to the co-amplification of the much shorter NS gene. The NS gene can be seen at the far right side of each of the line scans at approximately 1000 bp length. For all 9 viruses, the NS gene amplifies more strongly than any of the other anticipated products, indicating that in some cases the NS gene is likely outcompeting some of the other amplification products anticipated. FIG. 4 does show, however, successful amplification of NA in two N6 viruses, two N7 viruses, and one N9 virus. Because the samples tested are received as extracted RNA (and not as whole virus), the likely reason for failed amplification of some of the target gene segments in lanes 2, 7, and 8 is due to RNA degradation.

### Example 4: Initial Primer Testing, Singleplex Testing for all but NA Primers

Prior to full multiplexing, primers are tested in singleplex (or 3 primer pair multiplex in the case of the A/NA-targeted primers) reactions to demonstrate successful amplification. That is, all 3 primer pairs intended to amplify the A/NA gene (SEQ ID Nos: 3-8) are combined and tested in a multiplex fashion (given that 3 primer pairs are required to get successful amplification across the different NA subtypes), and the other primer pairs are tested in a singleplex fashion (as a single primer pair that is designed to amplify all subtypes of a gene segment). Each primer is present at 200 nM, and the following thermal cycling protocol is utilized: 48° C. for 20 min (reverse transcription), followed by 94° C. for 3 min (enzyme inactivation/activation), then 40 cycles of 94° C. for 10 sec, 51° C. for 30 sec, and 72° C. for 2 min.

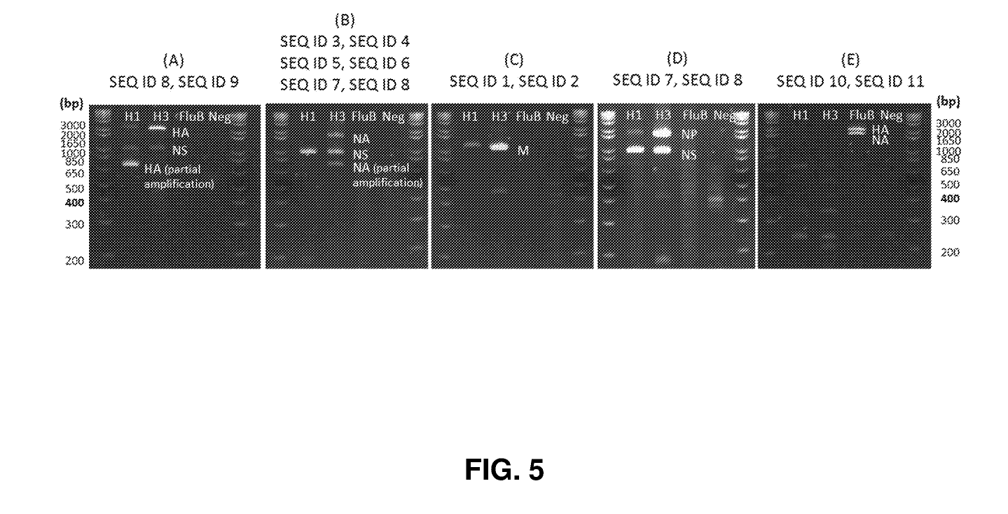

Given that a significant amount of optimization is likely necessary for a single tube multiplex formulation, a limited amount of testing is performed using these simplified mixtures of primers. FIG. 5 shows the results of agarose gel electrophoresis performed on the amplification products from an A/H1N1 virus (“H1”), an A/H3N2 virus (“H3”), an influenza B virus (“B”), and a negative control (“Neg”). Molecular weight markers are labeled with lengths in base pairs (bp) on either end and are also shown in numerous lanes of the gel throughout the figure. FIG. 5, panel (A) shows successful amplification of the A/HA gene segment as well as the A/NS gene segment. In addition, these primers amplify a shorter portion of the HA gene segment in A/H1 viruses. Panel (B) shows the amplification of the NA gene and NS gene segments. The NA gene for the A/H1N1 virus amplified only weakly, likely due to the fact that the NS gene is shorter and amplifies more efficiently. In addition, there is a shorter partial NA gene amplification product at ˜700 bp. Panel (C) shows successful amplification of the M gene segment for the influenza A viruses. Panel (D) shows co-amplification of both the NP and NS gene segments for the influenza A viruses with the primer pair composed of SEQ ID 7 and 8. Lastly, panel (E) shows successful co-amplification of both the HA and HA gene segments for an influenza B virus.

### Example 5: Multiplex Amplification

Optimization of the multiplexing conditions is accomplished by first optimizing the concentration of each primer in solution. Based on preliminary experiments, 400 nM is determined as a reasonable starting point for influenza primers, however, adjustments are made to certain primers in the mixture to counteract the fact that the gene segment being amplified is shorter than the other targets and therefore amplifies to a higher extent. This is the case for SEQ ID NOs: 1 and 2 that target the M gene segment; because the M gene segment is only ˜1000 bp in length, the primer concentration is reduced to 300 nM to minimize its amplification compared to some of the other targets. While it would be ideal to also reduce the concentration of the primers amplifying the NS gene segment (SEQ ID NOs: 7 and 8), this is not possible due to the fact that the same primer pair is used for the amplification of NA from N6, N7, and N9 subtypes, and lowering the concentration would also inhibit amplification of NA for these subtypes. It is also found that the optimal concentration of SEQ ID NO: 8 (the forward primer targeting HA) and SEQ ID NO: 9 (the reverse primer targeting HA, but also co-amplifying NS, NP, and NA for N6, N7, and N9) is 500 nM to allow HA to amplify to the fullest extent possible. Given the multifunctionality of SEQ ID NOs: 7 and 8, it is impossible to reduce the concentration of these primers to limit the amplification of the NS gene segment any further.

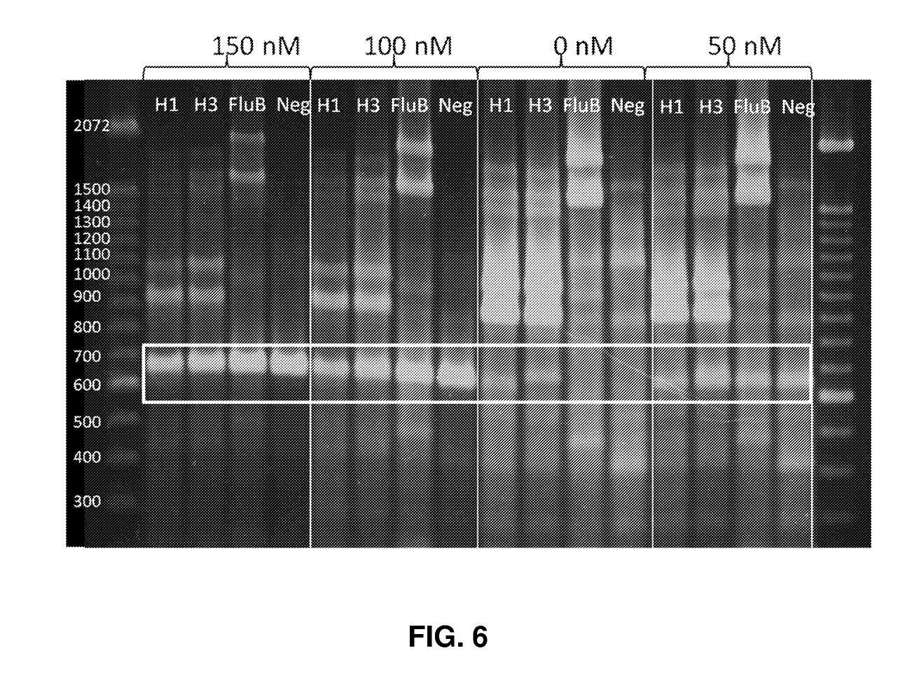

The 18s rDNA internal control primers (SEQ ID NOs: 12 and 13) are also optimized in terms of concentration. With the internal control, the concentration of primers is typically much lower than for the target(s) due to the potential for out-competition, as it is only imperative that the internal control amplify when influenza is not present. The goal therefore is to find a low concentration that provides consistent amplification, but that is not high enough to significantly inhibit the amplification of the target. To show the effect of changing concentration of the internal control primers on amplification of influenza, the concentration of the forward and reverse internal control primers are varied from 0 nM to 150 nM in 50 nM increments. FIG. 6 shows 1.2% agarose gel electrophoresis results of the four concentrations tested on an A/H1N1, A/H3N2, and B virus. Lanes 1 and 18 are the molecular weight marker. The internal control amplicon is the ˜650 bp product highlighted in the box, and the influenza amplicons are the higher molecular weight bands. These data indicate that inhibition of influenza amplification begins to occur at 100 nM. Based on these data and additional follow-up experiments, the concentration of internal control primers selected for the multiplexed mixture is 80 nM. One example of primer concentrations for a multiplex reaction is summarized in TABLE 4.

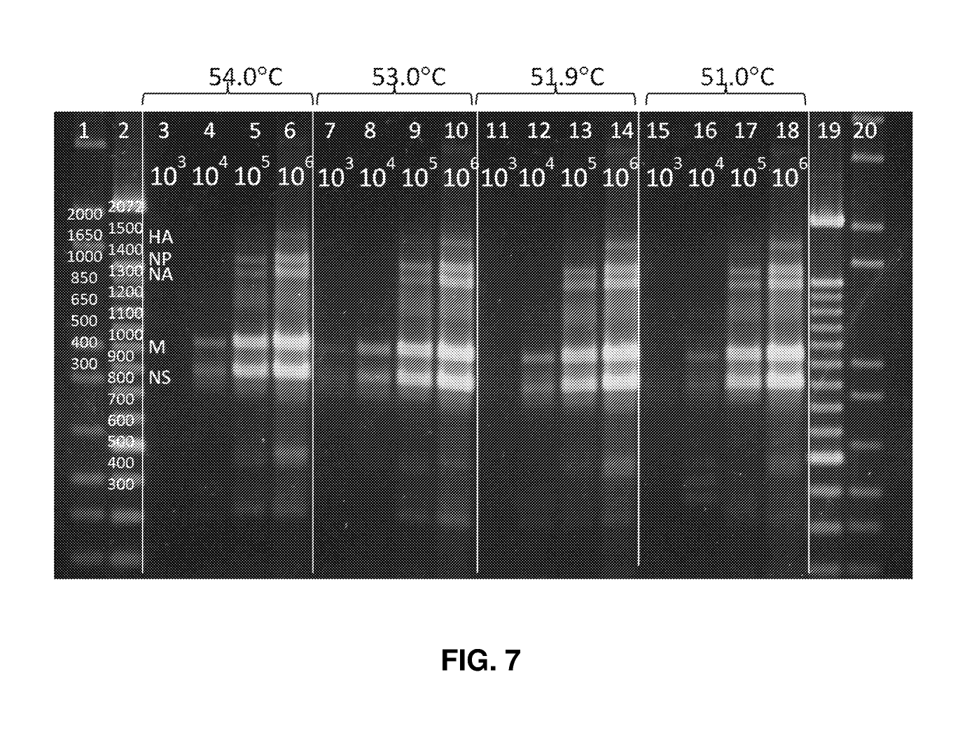

In addition to optimization of concentrations, optimal annealing temperature is also determined. FIG. 7 shows agarose gel electrophoresis results of the full primer mixture used to amplify A/Denver/1/57 (H1N1) at four different annealing temperatures: 54° C., 53° C., 52° C., and 51° C. Sixteen reactions are run (plus a no-template control), with the following template RNA concentrations tested at each annealing temperature: 1×10³, 1×10⁴, 1×10⁵, and 1×10⁶ copies/reaction. FIG. 7 shows successful amplification at all annealing temperatures tested, however, the lowest template concentration tested showed slightly better amplification with an annealing temperature of 53° C. Examples of calculated melting temperatures for the SEQ ID NOs: 1-13 relative to nucleotide sequences described by Hoffmann are summarized in TABLE 5. TABLE 5 illustrates that the carefully designed and configured primers of the instant invention have improved melting temperature characteristics, as indicated by the substantial decrease in standard deviation of melting temperature across all primers (e.g. 0.9° C. of the instant invention compared to 4.0° C. of the Hoffmann primer with the tag and 2.4° C. without the tag). The improvement in standard deviation of at least 60%: (2.4-0.9)/2.4 reflects the instant primers suitability for multiplex tests. In contrast, the higher melting temperature deviations in the art indicates those primers are not suited for a multiplex test, where one thermal cycle protocol is used to amplify multiple targets by multiple forward/revere primer pairs in parallel.

In particular, the primers of Hoffman comprise: (i) conserved sequencing tag at the 5′ end which are not used in the instant primers; (ii) the highly conserved 13 nt and 12 nt regions at the 5′ and 3′ of each segment; (iii) a two or three nt sequence at the end of each primer that makes the primer gene-specific. Important distinctions of the instant primers include regions where nucleotides are removed from the Hoffman primers to make the instant primers universal in nature, and regions where nucleotides are added to the instant primers to elevate melting temperatures so as to better match other primers of the primer set to achieve reliable multiplexing. Referring to TABLE 3, the underlined region is the universally conserved region at the start and end of each gene segment of the influenza virus. Nucleotides in bold italic represent differences in the conserved region between the instant primers and the Hoffman primers. Nucleotides in italics are nucleotides added to the instant primers from before the start codon to elevate primer melting temperature to better match the rest of the primers so as to facilitate multiplexing the primers under the same reaction condition.

The differences in the Hoffman primers and the primers of the instant invention are further reflected by the fact that Hoffman performed RT and PCR in two separate steps, and single universal primer was used for the RT step. Gene-specific primers were then subsequently used in a singleplex manner and were not combined in a single tube reaction. Furthermore, Hoffman used the primers for sequencing, so that no internal control was needed or developed. This is fundamentally different than the instant invention, where the entire primer mix is used in a single multiplex reaction, and RT-PCR is performed in a single step. In addition, the primer mix described herein includes an internal control for 18s RNA that is amplified in all eukaryotes as a check for specimen integrity. Primers described herein may also include a 5′ phosphorylation to allow subsequent digestion of the phosphorylated strand by lambda exonuclease for better downstream hybridization, as desired depending on the specifics of the detection or characterization system.

### Example 6: RT-PCR Reaction Setup and Execution with Final Primer Formulation and Conditions

10× primer mix is prepared by combining 544.0 μL of RNase/DNase-free water with the volumes shown in Table 2 of 100 μM stock primer solutions into a sterile screw cap vial. Primer mix is split into smaller aliquots and stored at −20° C. if not used immediately. Necessary components are thawed for RT-PCR reaction setup, and master mix prepared on ice in an appropriate template-free PCR setup area using appropriate workflow to prevent contamination. For each RT-PCR reaction to be run, 24.5 μL of 2× qScript XLT One-Step ToughMix and 1.4 μL of 25× qScript XLT One-Step Reverse Transcriptase (both from Quanta Biosciences) are combined, along with 3.5 μL of the above-prepared 10× primer mixture. If downstream detection via a streptavidin-coupled fluorophore is desired, also add 0.6 μL of biotin-16-aminoallyl-2′-dUTP to each reaction; otherwise, add 0.6 μL of RNase/DNase-free water. Volumes above were scaled to prepare multiple RT-PCR reactions at one time, aliquotting 30.0 μL of prepared reaction mixture into the appropriate number of properly-labeled PCR tubes. Note that appropriate positive controls and no-template negative control reactions were always included. In the laboratory area designated for handling extracted nucleic acid template, 5.0 μL of RNA template to be amplified was added to the appropriate tubes for a total RT-PCR reaction volume of 35.0 μL. Reaction tubes were placed in an appropriate thermocycler (such as a BioRad T100), the lid closed, and the following thermal profile performed: 48° C. for 20 min (reverse transcription), 94° C. for 3 min (enzyme inactivation/activation), 40 cycles of 94° C. for 10 sec, 53° C. for 30 sec, and 72° C. for 2 min.

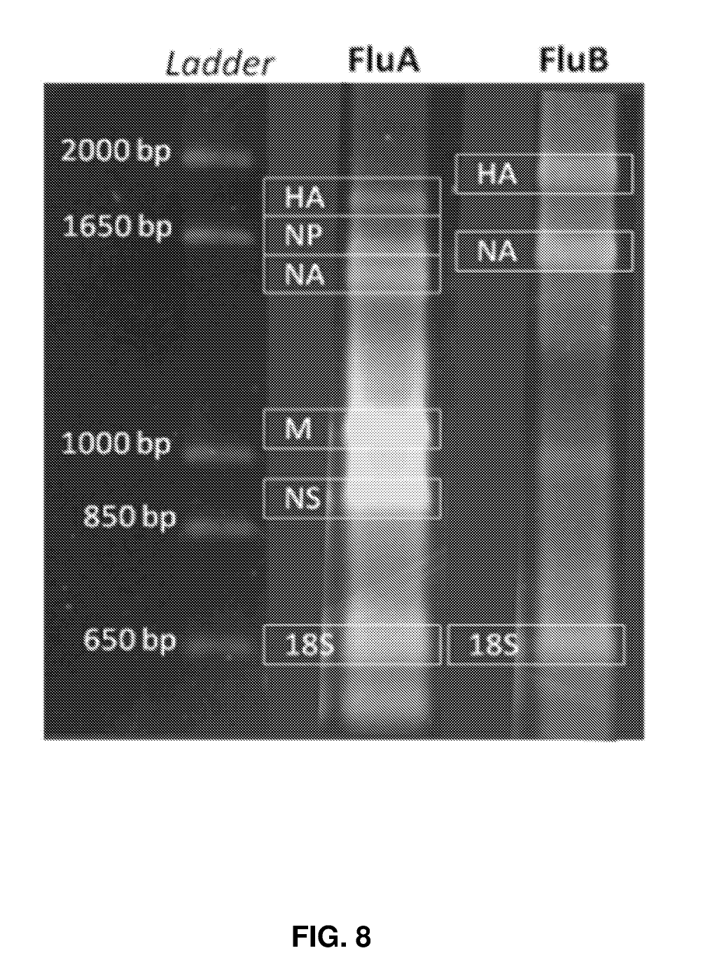

Amplified products are confirmed with agarose gel electrophoresis. The band lengths in Table 1 are expected, with the exact band length being potentially variable based on the particular virus subtype and/or strain. In addition, because biotin is sometimes incorporated into the PCR products (to allow various downstream detection techniques), the apparent length observed on the gel for the bands run longer on the gel than where the actual amplicon length would be expected due to the additional molecular weight of the biotin molecules. An example gel using the above protocol set up as described in Example 7 below is provided in FIG. 8.

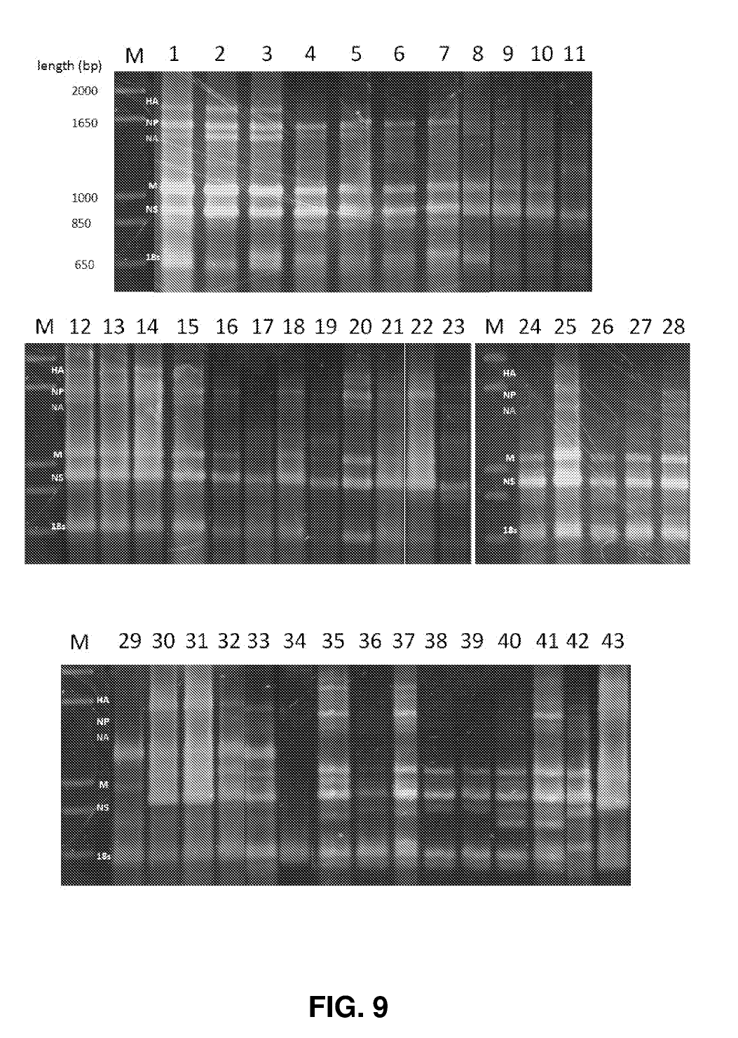

Additional virus samples or extracted RNA samples are tested under the above conditions, including eleven A/H3N2 samples (lanes 1-11), twelve A/H1N1 samples (lanes 12-23), five A/H5N1 samples (lanes 24-28), five H3N8 samples (lanes 29-33), four H10N7 samples (lanes 34-37), two H6N1 samples (lanes 38, 39), two H7N1 samples (lanes 40, 41), and one H1N2 sample (lane 42), and one H5N2 (lane 43), and agarose gel electrophoresis results are shown in FIG. 9. The molecular weight marker is also shown, marked as M at the top of the appropriate lanes, along with notations where the anticipated products are expected to appear on the gel. While not every product is visible for every sample tested, the majority of samples tested show amplification of the intended products. The non-human origin samples in lanes 24-43 are all tested as extracted RNA, and therefore could have been partially degraded at the time of testing due to long-term storage.

### Example 7: Gel Electrophoresis Protocol for Amplicon Detection

A 1.2% agarose gel is prepared by first dissolving 1.2 g of agarose in 100 mL of 1×TBE electrophoresis running buffer. The agarose solution is heated to boiling in a microwave with periodic swirling to achieve full dissolution, subsequently cooled to 50-60° C., and poured into an appropriate gel casting mold to cool. Once cooled, the comb and casting gates are carefully removed and the gel is placed into the tray and placed on the gel stage. Enough 1×TBE buffer is added to fully submerge the gel under ˜4-5 mm buffer. Amplified samples are prepared for gel loading by combining 8 μL of sample with 3 μL of 5× loading buffer. Samples are loaded onto the gel alongside a 100 bp TrackIt ladder, and run at 75 V for four hours. Stain the gel in a 1:10,000 dilution of SYBR gold prepared with 1×TBE running buffer by adding 15 μL of SYBR gold solution to 150 mL of 1×TBE (pH should be between 7.0 and 8.5). Add enough stain to completely cover the gel, and place gel and stain on an orbital shaker to agitate gently for 20 minutes. After staining (no destaining is needed), the gel is imaged, such as by placement on a sheet of plastic wrap on the UV gel imaging system and imaged.

### Example 8: Microarray Detection of Multiplex Amplification

DNA microarrays are an alternative detection method to agarose gel electrophoresis for detection of amplified PCR products produced with primer mixes such as that described herein. After nucleic acid extraction and multiplex amplification of influenza viral RNA from viral isolates using the final primer formulation and conditions described in Example 6, PCR products are fragmented by adding 10-20 μL of water and heating to 94° C. for 10 min. Fragmenting the DNA can improve hybridization by fragmenting the amplicons into smaller, single-stranded pieces of nucleic acid that then potentially hybridize to multiple different capture sequences on the microarray, as opposed to hybridization of one large, double-stranded amplicon that hybridizes to a single capture sequence on the microarray. Fragmented amplicons are hybridized to a DNA microarray containing important influenza target oligonucleotides specific to many subtypes of influenza, and subsequently fluorescently-labeled for downstream optical detection. Microarrays are then imaged using a fluorescence microarray imaging instrument, and the signals from the capture sequences on the DNA microarray confirm that amplification was successful.

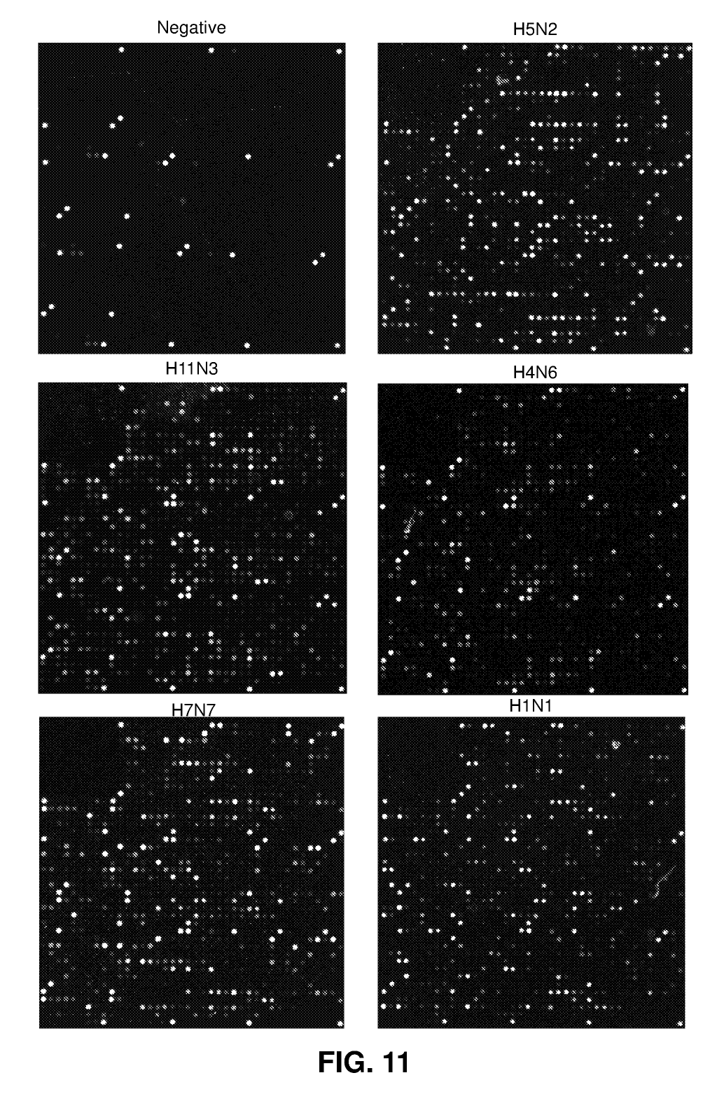

Over 1200 clinical samples and influenza viral isolates have been successfully amplified via multiplex RT-PCR utilizing SEQ ID Nos: 1-13 and the protocol described in Example 6 (followed by downstream microarray detection. FIG. 11 shows examples of microarray images for various influenza A subtypes (H5N2, H11N3, H4N6, H7N7, H1N1) and a non-template control (top left panel labeled “Negative”) that does not contain influenza for reference. The microarray detection reflects successful amplification using the universal primer formulation, as evidenced by generation of signal intensities above background from all amplified influenza A subtypes listed in TABLE 6. Included in the 58 influenza A subtypes successfully amplified, a wide diversity of different strains and host species/species of isolation are represented.

In addition, a wide variety of influenza B viruses from both the Yamagata and Victoria lineages have been successfully amplified via multiplex RT-PCR utilizing the final primer formulation described herein followed by detected using an influenza-specific DNA microarray for detection. Thirty-two influenza B strains that span 73 years of isolation, both influenza B lineages, and represent broad geographic diversity have been amplified by the universal multiplex primer formulation described herein and subsequently detected by a microarray. The strains detected are detailed in TABLE 7. Further, over 120 influenza B clinical specimens (collected from 2008 to 2014, from sources in the US and Sri Lanka) have been amplified using the universal primer set and detected on a microarray. These data demonstrate that the primer formulation presented herein broadly amplifies influenza viruses of a wide variety of types and subtypes, with successful universal amplification of influenza viruses from avian, human, swine, equine, canine, and other species.

### Example 9: Kits

Also provided herein are kits to allow a user to diagnose and/or identify influenza, such as by amplification of relevant amplicons indicative of influenza. Such kits contain at least one container of primers, including a single container comprising all the primers required in a single multiplex reaction. Alternatively, the primers may be provided in individual containers which can then be mixed together for a single multiplex reaction. Kits can optionally provide instructions for carrying out disclosed methods of amplifying influenza gene segments, including for diagnosing and identifying influenza infection. In particular examples, the kits additionally include reagents useful in generating the amplicon, such at least one type of thermal-stable DNA polymerase, nucleotides and/or buffers necessary for PCR amplification of a DNA sequence.

Certain kits include reagents useful for identification of specific types or strains of influenza. Examples of such kits include at least one pair of primers specific for a gene of influenza B to assist in diagnosis or identification of influenza A or B.

The materials provided in such kits may be provided in any form practicable, such as suspended in an aqueous solution or as a freeze-dried or lyophilized powder, for instance. Kits according to this disclosure can also include instructions, usually written instructions, to assist the user in carrying out the detection and identification methods disclosed herein. Such instructions can optionally be provided on a computer readable medium or as a link to an internet website where instructions are provided.

The container(s) in which the materials are supplied can be any container that is capable of holding the material, such as microfuge tubes, ampules, or bottles. In some applications, the primers, thermal-stable nucleic acid polymerase(s), restriction endonuclease(s), or other reagent mixtures useful for diagnosis and identification of influenza may be provided in pre-measured single use amounts in individual, typically disposable, tubes, microtiter plates, or equivalent containers. The containers may also be compatible with a specific automated liquid handling apparatus.

The amount of a reagent supplied in the kit can be any appropriate amount, depending for instance on the market to which the product is directed. For instance, if the kit is adapted for research or clinical use, the amount of each reagent, such as the primers, thermal-stable nucleic acid polymerase(s), or restriction endonuclease(s) would likely be an amount sufficient for multiple screening assays. In other examples where the kit is intended for high throughput industrial use, the amounts could be sufficiently increased to accommodate multiple hundreds of assays.

### STATEMENTS REGARDING INCORPORATION BY REFERENCE AND VARIATIONS

All references throughout this application, for example patent documents including issued or granted patents or equivalents; patent application publications; and non-patent literature documents or other source material; are hereby incorporated by reference herein in their entireties, as though individually incorporated by reference, to the extent each reference is at least partially not inconsistent with the disclosure in this application (for example, a reference that is partially inconsistent is incorporated by reference except for the partially inconsistent portion of the reference).

The terms and expressions which have been employed herein are used as terms of description and not of limitation, and there is no intention in the use of such terms and expressions of excluding any equivalents of the features shown and described or portions thereof, but it is recognized that various modifications are possible within the scope of the invention claimed. Thus, it should be understood that although the present invention has been specifically disclosed by preferred embodiments, exemplary embodiments and optional features, modification and variation of the concepts herein disclosed may be resorted to by those skilled in the art, and that such modifications and variations are considered to be within the scope of this invention as defined by the appended claims. The specific embodiments provided herein are examples of useful embodiments of the present invention and it will be apparent to one skilled in the art that the present invention may be carried out using a large number of variations of the devices, device components, methods steps set forth in the present description. As will be obvious to one of skill in the art, methods and devices useful for the present methods can include a large number of optional composition and processing elements and steps.

When a group of substituents is disclosed herein, it is understood that all individual members of that group and all subgroups, are disclosed separately. When a Markush group or other grouping is used herein, all individual members of the group and all combinations and subcombinations possible of the group are intended to be individually included in the disclosure.

Every formulation or combination of components described or exemplified herein can be used to practice the invention, unless otherwise stated.

Whenever a range is given in the specification, for example, a temperature range, a time range, or a composition or concentration range, all intermediate ranges and subranges, as well as all individual values included in the ranges given are intended to be included in the disclosure. It will be understood that any subranges or individual values in a range or subrange that are included in the description herein can be excluded from the claims herein.

All patents and publications mentioned in the specification are indicative of the levels of skill of those skilled in the art to which the invention pertains. References cited herein are incorporated by reference herein in their entirety to indicate the state of the art as of their publication or filing date and it is intended that this information can be employed herein, if needed, to exclude specific embodiments that are in the prior art. For example, when composition of matter are claimed, it should be understood that compounds known and available in the art prior to Applicant's invention, including compounds for which an enabling disclosure is provided in the references cited herein, are not intended to be included in the composition of matter claims herein.

As used herein, “comprising” is synonymous with “including,” “containing,” or “characterized by,” and is inclusive or open-ended and does not exclude additional, unrecited elements or method steps. As used herein, “consisting of” excludes any element, step, or ingredient not specified in the claim element. As used herein, “consisting essentially of” does not exclude materials or steps that do not materially affect the basic and novel characteristics of the claim. In each instance herein any of the terms “comprising”, “consisting essentially of” and “consisting of” may be replaced with either of the other two terms. The invention illustratively described herein suitably may be practiced in the absence of any element or elements, limitation or limitations which is not specifically disclosed herein.

One of ordinary skill in the art will appreciate that starting materials, biological materials, reagents, synthetic methods, purification methods, analytical methods, assay methods, and biological methods other than those specifically exemplified can be employed in the practice of the invention without resort to undue experimentation. All art-known functional equivalents, of any such materials and methods are intended to be included in this invention. The terms and expressions which have been employed are used as terms of description and not of limitation, and there is no intention that in the use of such terms and expressions of excluding any equivalents of the features shown and described or portions thereof, but it is recognized that various modifications are possible within the scope of the invention claimed. Thus, it should be understood that although the present invention has been specifically disclosed by preferred embodiments and optional features, modification and variation of the concepts herein disclosed may be resorted to by those skilled in the art, and that such modifications and variations are considered to be within the scope of this invention as defined by the appended claims.

TABLE 1

| Target | Amplicon size (bp) |
|---|---|
| 18s internal control | ~650 |
| Flu A NS gene segment | ~900 |
| Flu A M gene segment | ~1030 |
| Flu A NA gene segment | ~1450 |
| Flu A NP gene segment | ~1550 |
| Flu B NA gene segment | ~1550 |
| Flu A HA gene segment | ~1750 |
| Flu B HA gene segment | ~1890 |

TABLE 2

| Seq ID No. | Volume added (μL) | Concentration in 10x mix |
|---|---|---|
| 1 | 30.0 | 3 μM |
| 2 | 30.0 | 3 μM |
| 3 | 40.0 | 4 μM |
| 4 | 40.0 | 4 μM |
| 5 | 40.0 | 4 μM |
| 6 | 40.0 | 4 μM |
| 7 | 40.0 | 4 μM |
| 8 | 50.0 | 5 μM |
| 9 | 50.0 | 5 μM |
| 10 | 40.0 | 4 μM |
| 11 | 40.0 | 4 μM |
| 12 | 8.0 | 0.8 μM |
| 13 | 8.0 | 0.8 μM |

TABLE 3
Primer Sequence Summary

| SEQ | | | |
|---|---|---|---|
| ID | FC8G PRIMERS | | |
| NO: | TARGET(S) | ID | SEQUENCE 5′→3′ |
| 1 | M | M 5′P degen (for) | AGAGC [symbol] AAAGCAG |
| | | | GTAG |
| 2 | | M (rev) | GGGAGTAGAAACAA |
| | | | GGTAG |
| 3 | NA (only N1, | N1, N2, N4, N5, N8 | AGAGCAAAAGCAGG |
| | N2, N4,N5, | (for) 5′P | AG |
| 4 | N8) | N1, N2, N4, N5, N8 | GGGAGTAGAAACAA |
| | | (rev) | GGAG |
| 5 | NA (only N3) | N3 (for) 5′P | AGAGCAAAAGCAGG |
| | | | TG |
| 6 | N3 (rev) | | GGGAGTAGAAACAA |
| | | | GGTG |
| 7 | NA (only N6, | NS ,NP, N6, N7, N9 | GAGC [symbol] AAAGCAG |
| | N7, N9), NS, | (for) 5′P | GGT |
| 8 | NP | NS, NP, HA, N6, | GGGAGTAGAAACA |
| | | N7, N9 (rev) | AGGGT |
| 9 | HA | HA (for) 5′P | AGCAAAAGCAGGG |
| | | | G |
| 8 | | NS, NP, HA, N6, | GGGAGTAGAAACA |
| | | N7, N9 (rev) | AGGGT |
| 10 | FluB | HA and NA (for) | AGCAGAAGCAGAG |
| | | 5′P | CAT |
| 11 | (HA, NA) | HA and NA (rev) | CAGTAGTAACAAG |
| | | | AGCATTT |
| 12 | 18s | 18s (for) 5′P | CCTGAGAAACGGC |
| 13 | Control | 18s Reverse | TAC |
| | | 800-815 | TTATGGTCGGAAC |
| | | | TACG |

TABLE 4

| SEQ ID | Concentration used | Lower Limit | Upper Limit |
|---|---|---|---|
| NO: | (nM) | (nanomolar) | (nanomolar) |
| 1 | 300 | 225 | 375 |
| 2 | 300 | 225 | 375 |
| 3 | 400 | 300 | 500 |
| 4 | 400 | 300 | 500 |
| 5 | 400 | 300 | 500 |
| 6 | 400 | 300 | 500 |
| 7 | 400 | 300 | 500 |
| 8 | 500 | 375 | 625 |
| 9 | 500 | 375 | 625 |
| 10 | 400 | 300 | 500 |
| 11 | 400 | 300 | 500 |
| 12 | 80 | 60 | 100 |
| 13 | 80 | 60 | 100 |

TABLE 5

| | | | | Tm | Tm |
|---|---|---|---|---|---|
| | | Tm | | (with | (without |
| ID | Our Sequences | (° C)* | Hoffmann Primers | tag**) | tag**) |
| 1 | AGAGCRAAAGCAGGTAG | 52.4 | TATTCGTCTCAGGGAGCAAAAGCAGGTAG | 71.4 | 46.4 |
| 2 | GGGAGTAGAAACAAGGTAG | 51.1 | ATATCGTCTCGTATTAGTAGAAACAAGGTAGTTTTT | 65.1 | 49.3 |
| 3 | AGAGCAAAAGCAGGAG | 51.7 | TATTGGTCTCAGGGAGCAAAAGCAGGAGT | 72.7 | 48.7 |
| 4 | GGGAGTAGAAACAAGGAG | 51.8 | ATATGGTCTCGTATTAGTAGAAACAAGGAGTTTTTT | 66.3 | 51.6 |
| 5 | AGAGCAAAAGCAGGTG | 52.6 | | | |
| 6 | GGGAGTAGAAACAAGGTG | 52.6 | | | |
| 7 | GAGCRAAAGCAGGGT | 53.8 | TATTCGTCTCAGGGAGCAAAAGCAGGGTG | 75.2 | 53.4 |
| | | | TATTCGTCTCAGGGAGCAAAAGCAGGGTA | 73.1 | 50.0 |
| 8 | GGGAGTAGAAACAAGGGT | 53.7 | ATATCGTCTCGTATTAGTAGAAACAAGGGTATTTTT | 66.2 | 51.5 |
| 9 | AGCAAAAGCAGGGG | 53.3 | TATTCGTCTCAGGGAGCAAAAGCAGGGGG | 75.2 | 53.3 |
| 8 | GGGAGTAGAAACAAGGGT | | ATATCGTCTCGTATTAGTAGAAACAAGGGTGTTTT | 67.6 | 52.9 |
| 10 | AGCAGAAGCAGAGCAT | 52.1 | | | |
| 11 | CAGTAGTAACAAGAGCATTT | 49.5 | | | |
| 12 | CCTGAGAAACGGCTAC | 51.9 | | | |
| 13 | TTATGGTCGGAACTACG | 52.4 | | | |
| | Average of all Flu | 52.6 ± | | 70.3 ± | 50.8 ± |
| | A primers | 0.9 | | 4.0 | 2.4 |

*Calculated using a nearest neighbor approach in OligoAnalyzer 1.0.3 software. Where a mixed base position exists, the Tm listed is the average.
**“tag” means sequencing tag in green text.

TABLE 6
Influenza A Subtypes Successfully
Amplified and Detected on a Microarray

| Subtype | Host(s) |
|---|---|
| H1N1 | Avian, Human, Swine |
| H1N2 | Avian, Swine |
| H1N3 | Avian |
| H1N8 | Avian |
| H2N1 | Lab Reassortant |
| H2N2 | Human |
| H2N3 | Avian |
| H2N9 | Avian |
| H3N1 | Lab Reassortant |
| H3N2 | Canine, Human, Swine, |
| H3N6 | Avian |
| H3N7 | Lab Reassortant |
| H3N8 | Avian, Canine, Equine |
| H3N9 | Avian |
| H4N2 | Avian |
| H4N3 | Avian |
| H4N6 | Avian |
| H4N8 | Unknown |
| H5N1 | Avian, Human |
| H5N2 | Avian |
| H5N3 | Lab Reassortant |
| H5N4 | Avian |
| H5N6 | Avian |
| H5N7 | Avian |
| H5N8 | Avian |
| H5N9 | Avian |
| H6N1 | Avian |
| H6N2 | Avian |
| H6N4 | Avian |
| H6N5 | Avian |
| H6N8 | Unknown |
| H7N1 | Avian |
| H7N2 | Avian |
| H7N3 | Avian |
| H7N4 | Avian |
| H7N5 | Avian |
| H7N6 | Avian |
| H7N7 | Avian, Equine, Human, Seal |
| H7N8 | Avian |
| H7N9 | Avian, Human |
| H8N4 | Avian |
| H9N2 | Avian, Human |
| H9N7 | Avian |
| H9N9 | Avian |
| H10N1 | Avian |
| H10N2 | Avian |
| H10N7 | Avian |
| H10N8 | Avian |
| H11N1 | Unknown |
| H11N2 | Avian |
| H11N3 | Avian |
| H11N6 | Avian |
| H11N9 | Avian |
| H12N5 | Avian |
| H13N6 | Avian |
| H14N5 | Avian |
| H15N9 | Avian |
| H16N3 | Avian |

TABLE 7
Influenza B Strains Successfully Amplified and Detected on a Microarray

| Strain | Lineage |
|---|---|
| B/Lee/1940 | N/A |
| B/Great Lakes/1739/1954 | N/A |
| A/Denver/1/1957 | N/A |
| B/Taiwan/2/1962 | N/A |
| A/Aichi/2/1968 | N/A |
| B/Harbin/07/1994 | Yamagata |
| B/Memphis/20/1996 | Yamagata |
| B/Rochester/20/1996 | Yamagata |
| B/Perth/211/2001 | Yamagata |
| B/Florida/07/2004 | Yamagata |
| B/Malaysia/2506/2004 | Victoria |
| B/Florida/02/2006 | Victoria |
| B/Florida/04/2006 | Yamagata |
| B/Victoria/304/2006 | Victoria |
| B/Bangladesh/3333/2007 | Yamagata |
| B/Brisbane/03/2007 | Yamagata |
| B/Chongqing/Yongchuan18/2007 | Yamagata |
| B/Pennsylvania/07/2007 | Yamagata |
| B/Brisbane/60/2008 | Victoria |
| B/Bangladesh/9673/2009 | Yamagata |
| B/Finland/39/2010 | Yamagata |
| B/Wisconsin/01/2010 | Yamagata |
| B/Cambodia/30/2011 | Victoria |
| B/Fujian/Gulou/1553/2011 | Yamagata |
| B/Georgia/01/2011 | Victoria |
| B/North Carolina/03/2011 | Victoria |
| B/Nevada/03/2011 | Victoria |
| B/Texas/06/2011 | Yamagata |
| B/Massachusetts/02/2012 | Yamagata |
| B/New Jersey/01/2012 | Victoria |
| B/Phuket/3073/2013 | Yamagata |
| B/Texas/02/2013 | Victoria |

We claim:

1. A universal primer set for amplification of whole gene segments HA, NA, M, NS and NP from an influenza A virus in a single multiplex reaction, the universal primer set comprising isolated and purified nucleic acids of SEQ ID NOs:1-9 for targeting the whole gene segments.

2. The universal primer set of claim 1, further comprising a plurality of nucleic acid primers for amplification of whole gene segments HA and NA from an influenza B virus in said single multiplex reaction, the plurality of nucleic acid primers comprising isolated and purified nucleic acids of SEQ ID NOs:10-11.

3. The universal primer set of claim 1, further comprising a plurality of nucleic acid control primers for amplification of a control 18s gene in said single multiplex reaction, the plurality of nucleic acid control primers comprising isolated and purified nucleic acids of SEQ ID NOs:12-13.

4. The universal primer set of claim 2, further comprising a plurality of nucleic acid control primers for amplification of a control 18s gene in said single multiplex reaction, the plurality of nucleic acid control primers comprising isolated and purified nucleic acids of SEQ ID NOs:12-13.

5. The universal primer set of claim 1, provided in a single mixture for a single multiplex reaction amplification.

6. A universal primer set for amplification of whole gene segments HA and NA from an influenza B virus in a single multiplex reaction, the universal primer set comprising isolated and purified nucleic acids of SEQ ID NOs:10-11 for targeting the whole gene segments and SEQ ID NOs:12-13 for amplification of a control 18s gene.

7. The universal primer set of any of claims 1-6, wherein individual nucleic acids have a selected concentration for substantially simultaneous amplification of all influenza viruses within a single sample, wherein the concentration of each primer is within 25% of a concentration value selected from one or more of:
SEQ ID NO:1 having the concentration value of 300 nM;
SEQ ID NO:2 having the concentration value of 300 nM;
SEQ ID NO:3 having the concentration value of 400 nM;
SEQ ID NO:4 having the concentration value of 400 nM;
SEQ ID NO:5 having the concentration value of 400 nM;
SEQ ID NO:6 having the concentration value of 400 nM;
SEQ ID NO:7 having the concentration value of 400 nM;
SEQ ID NO:8 having the concentration value of 500 nM;
SEQ ID NO:9 having the concentration value of 500 nM;
SEQ ID NO:10 having the concentration value of 400 nM;
SEQ ID NO:11 having the concentration value of 400 nM;
SEQ ID NO:12 having the concentration value of 80 nM;
SEQ ID NO:13 having the concentration value of 80 nM; or
any combination thereof.

8. The universal primer set of any of claims 1-6 for influenza A characterization, wherein each individual nucleic acid has a melting temperature that is substantially matched to every other individual nucleic acid melting temperature for a multiplex reaction, the melting temperature characterized by one or more of:
a minimum primer melting temperature that is between about 51.0-52.0° C. and a maximum primer melting temperature is between about 53.7-53.9° C.;
an average melting temperature that is between about 52.5-52.7° C.; or
a standard deviation of all the melting temperatures that is less than about 1° C.

9. An isolated and purified nucleic acid for use in influenza detection, selected from the group consisting of:
SEQ ID NO:1 and/or SEQ ID NO:2 for targeting an influenza A whole M gene, wherein the nucleotide R is a purine, or a sequence that is at least 80% identical thereto;
SEQ ID NO:3 and/or SEQ ID NO:4 for targeting an influenza A whole NA gene (subtype N1, N2, N4, N5, N8), or a sequence that is at least 80% identical thereto;
SEQ ID NO:5 and/or SEQ ID NO:6 for targeting an influenza A whole NA gene (subtype N3), or a sequence that is at least 80% identical thereto;
SEQ ID NO: 7 and/or SEQ ID NO:8 for targeting an influenza A whole NA, NS, NP gene (subtype N6, N7, N9) wherein the nucleotide R is a purine, or a sequence that is at least 80% identical thereto; and
SEQ ID NO: 9: and/or SEQ ID NO:8 for targeting an influenza A whole HA gene, or a sequence that is at least 80% identical thereto.

10. The isolated and purified nucleic acid of claim 9, further comprising at least one additional nucleic acid for targeting an influenza B gene, comprising:
SEQ ID NO:10 (AGCAGAAGCAGAGCAT) and/or SEQ ID NO:11 (CAGTAGTAACAAGAGCATTT) for targeting an influenza B whole HA and NA gene, or a sequence that is at least 80% identical thereto.

11. The isolated and purified nucleic acid of claim 9 or 10, further comprising at least one additional nucleic acid that is a control, comprising:
SEQ ID NO: 12 (CCTGAGAAACGGCTAC) and/or SEQ ID NO:13 (TTATGGTCGGAACTACG) for targeting a gene coding for 18s rRNA as a control, or a sequence that is at least 80% identical thereto.

12. The isolated and purified nucleic acid of any of claims 9-11, further comprising phosphorylation at a 5′-end of the nucleic acid.

13. The isolated and purified nucleic acid of claim 9, except for SEQ ID NO:9.

14. The isolated and purified nucleic acid of claim 9 for targeting HA, NA and M of influenza A, comprising: SEQ ID NOs:1-9.

15. The isolated and purified nucleic acid of claim 9, comprising: SEQ ID NO: 2.

16. A plurality of isolated and purified nucleic acids comprising: SEQ ID NOs:1-13.

17. The plurality of isolated and purified nucleic acids of claim 16, wherein each nucleic acid is configured for use in a single multiplex RT-PCR.

18. A method for determining the presence or absence of influenza virus in a sample, the method comprising the steps of:
providing a universal primer cocktail for amplification of influenza whole gene targets comprising:
one or more influenza A gene segments HA, NA, M, NS and NP; and/or
one or more influenza B gene segments HA and NA;
contacting a sample with said universal primer cocktail;
performing RT-PCR on said sample in contact with said universal primer cocktail in a single multiplex reaction step; and
detecting amplified products from said performing RT-PCR step, thereby determining the presence or absence of influenza virus.

19. The method of claim 18, wherein the universal primer cocktail further comprises primers for amplification of an 18s control.

20. The method of claim 19, wherein the universal primer cocktail comprises SEQ ID NOs:1-13, or sequences that are at least 80% identical thereto.

21. The method of claim 20, wherein the performing step comprises:
a reverse transcription (RT) step at a temperature of between 47° C. to 49° C. for a time of between 18 minutes and 22 minutes;
a RT inactivation and polymerase activation step at a temperature of between 93° C. and 95° C. for between 2 min and 4 min;
a polymerase chain reaction (PCR) step for a PCR cycle number, wherein said PCR cycle number is greater than or equal to 35 cycles and less than or equal to 45 cycles.

22. The method of claim 21, for use in detecting influenza B, seasonal A/H1N1, seasonal A/H3N2, and non-seasonal A influenza strains.

23. A kit for detecting an influenza virus comprising:
any combination of the primers of claims 1-16, wherein the primers comprise a plurality of forward primers and a plurality of reverse primers for amplification of a plurality of influenza whole gene segments selected from the group consisting of influenza HA, NA, M, NS and NP; and
reagents for amplification by RT-PCR of said plurality of whole gene segments in a single multiplex reaction.

24. A kit for carrying out any of the methods of claims 18-22 comprising:
at least one container containing a plurality of distinct oligonucleotide primers having individual sequences that consist of SEQ ID NOs: 1-13;
at least one container containing reagents for performing RT-PCR for amplification of whole gene segments for influenza HA, NA, M, NS, and NP; and
instructions for performing RT-PCR using the oligonucleotide primers and reagents in a single multiplex reaction and detecting amplification products.


SEQUENCE LISTING (13 sequences)

SEQ ID NO: 1 - DNA, 17 bp, Artificial Sequence
  Synthetic construct
  r (6)..(6): r is a purine
agagcraaagcaggtag

SEQ ID NO: 2 - DNA, 19 bp, Artificial Sequence
  Synthetic construct
gggagtagaaacaaggtag

SEQ ID NO: 3 - DNA, 16 bp, Artificial Sequence
  Synthetic construct
agagcaaaagcaggag

SEQ ID NO: 4 - DNA, 18 bp, Artificial Sequence
  Synthetic construct
gggagtagaaacaaggag

SEQ ID NO: 5 - DNA, 16 bp, Artificial Sequence
  Synthetic construct
agagcaaaagcaggtg

SEQ ID NO: 6 - DNA, 18 bp, Artificial Sequence
  Synthetic construct
gggagtagaaacaaggtg

SEQ ID NO: 7 - DNA, 15 bp, Artificial Sequence
  Synthetic construct
  r (5)..(5): r is a purine
gagcraaagcagggt

SEQ ID NO: 8 - DNA, 18 bp, Artificial Sequence
  Synthetic construct
gggagtagaaacaagggt

SEQ ID NO: 9 - DNA, 14 bp, Artificial Sequence
  Synthetic construct
agcaaaagcagggg

SEQ ID NO: 10 - DNA, 16 bp, Artificial Sequence
  Synthetic construct
agcagaagcagagcat

SEQ ID NO: 11 - DNA, 20 bp, Artificial Sequence
  Synthetic construct
cagtagtaacaagagcattt

SEQ ID NO: 12 - DNA, 16 bp, Artificial Sequence
  Synthetic construct
cctgagaaacggctac

SEQ ID NO: 13 - DNA, 17 bp, Artificial Sequence
  Synthetic construct
ttatggtcggaactacg